(19) United States
(12) Patent Application Publication
(10) Pub. No.: US 2008/0092250 A1
(43) Pub. Date: Apr. 17, 2008

(54) Activity-induced pain in animal model of osteoarthritis

(76) Inventors: Prasant Chandran, Grayslake, IL (US); Madhavi V. Pai, Schaumburg, IL (US); Gin C. Hsieh, Long Grove, IL (US); Marie P. Honore, Evanston, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/546,714

(22) Filed: Oct. 12, 2006

(51) Int. Cl. A01K 67/027

(52) U.S. Cl. 800/9; 800/14

(57) ABSTRACT

The present invention presents a novel activity-induced pain behavior in ostoeoarthritic animals, useful for pharmacological characterization of potential analgesics.

### BACKGROUND OF THE INVENTION

Osteoarthritis (OA) or degenerative joint disease is by far the most common type of arthritis and afflicts millions of people worldwide. Pain worsened by weight bearing activity is one of the cardinal symptoms of OA and is the leading cause of disability and impairment in the quality of life due to functional limitations (ACR subcommittee of OA guidelines, 2000; O'Reilly et al., 1998). In the US, the percentage of people with OA-induced functional limitation is projected to increase from 2.8 in 1990 to 3.6% of the population in 2020 (Yasmin et al., 2000). Therefore, pain management is a challenging but critical cornerstone for the pharmacotherapy of OA, which in effect facilitates maintaining or improving joint mobility and minimizing functional impairment.

Relief from chronic joint pain secondary to osteoarthritis is often refractory to currently available pharmacological interventions (Kidd, 2006). Complete pain relief is elusive, posing a challenge for the clinicians. The pain in OA is complex as its etiology is often from multiple sources and can be either inflammatory or non-inflammatory in origin (Pinals, 1996). Clinically, the patients afflicted with OA describe their joint pain as being a deep and dull ache that is exacerbated with motion or activity. Activity-induced pain is a characteristic early symptom however, with the progression of the disease, a continuous aching pain or pain at rest is the main clinical presentation (Sinkov and Cymet, 2003). Thus, activity-induced pain is clinically relevant to the early stage OA and measuring pain during activity should be more of a sensitive measure and a better early predictor of pharmacological efficacy than when pain is measured at rest in a preclinical animal model.

Recent clinical studies have reported a difference in pharmacological profile for relief of pain at rest vs. activity-induced pain in OA patients (Petrella et al., 2002). Therefore, a thorough understanding of the mechanisms of actions of currently used analgesics and their utility in treating the different qualities of OA pain is vital.

Evaluation of hind limb grip force in animal models of OA may reproduce clinically observed symptoms of movement-induced pain in OA patients. There have been reports of several animal models of OA that are commonly used to evaluate the pathogenesis and potential therapeutic modulation of the disease (reviewed by Bendele, 2001). Intra-articular administration (i.a.) of sodium monoiodoacetate (MIA) in rodents causes a disruption of the articular cartilage by inhibiting chondrocyte metabolism (van der Kraan et al., 1992; Guingamp et al., 1997). The progressive joint degeneration and functional impairment following MIA injection mimics osteoarthritis induced joint destruction and activity limitation in the clinical population (Guingamp et al., 1997; Clarke et al., 1997). There are no experimental models that can be used in the assessment of activity-induced pain, a clinically relevant early onset symptom of OA. The present invention presents a novel activity-induced pain behavior in ostoeoarthritic animals, useful for pharmacological characterization of potential analgesics.

### BRIEF DESCRIPTION OF THE FIGURES

FIG. 1. (A) Time course of changes in body weight in monoiodoacetate (MIA) and saline injected rats. (B) Time course of changes in hindlimb grip force (GF) in MIA and saline injected rats.

FIG. 2. (A) Effects of morphine (s.c.) and tramadol (i.p.) on GF in rats 20 days following injection of MIA. (B) Effects of morphine (s.c.) and haloperidol (i.p.) on GF in naïve rats.

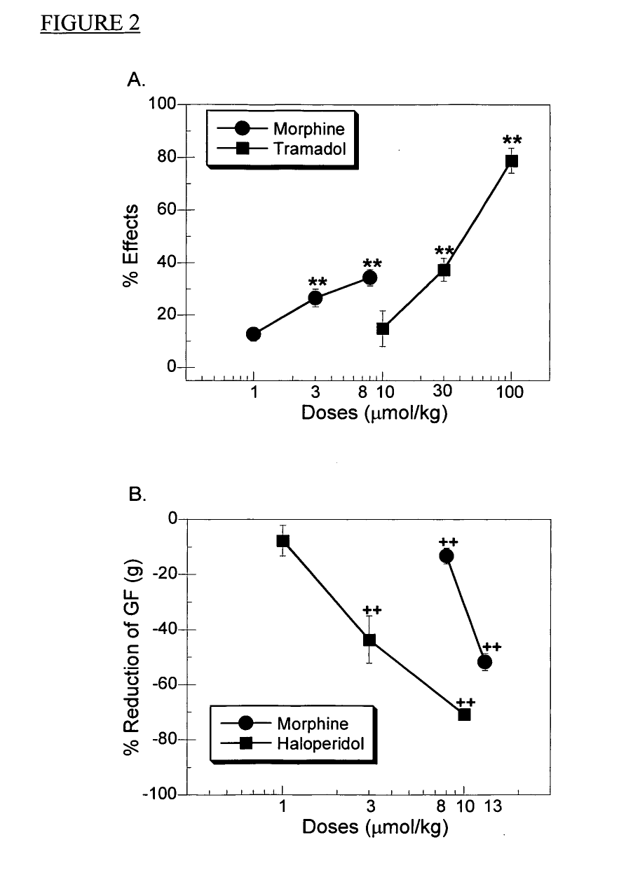

FIG. 3. (A) Effects of acetaminophen (p.o.) and NSAIDs: diclofenac (p.o.), ibuprofen (p.o.), celecoxib (p.o.) and indomethacin (p.o.) on GF in rats 20 days following injection of MIA. (B) Chronic dosing of celecoxib (10 and 30 μmol/kg p.o. b.i.d.) for 12 days beginning 7 days after MIA injection

FIG. 4. Effects of gabapentin (p.o.) and lamotrigine (p.o.) on GF in rats 20 days following injection of MIA.

### DETAILED DESCRIPTION OF THE INVENTION

The present invention provides a method of detecting and measuring activity-induced pain in an animal model in which ostheoarthritic-like changes have been induced There are several ways to induce ostheoarthritic-like changes. Osteoarthritic like changes can be induced by surgery such as in the cruciate ligament transection Models (Satsuma et al., 1996), partial medial meniscectomy in rats (Fernihough et al., 2005), extra-articular myectomy and tenotomy-induced hip OA in guinea pigs (Layton et al., 1987). Osteoarthritic like changes can also be chemically-induced by corticosteroids in the knee joints of mice (Silberberg et al., 1966), articular injections of proteolytic enzymes such as papain, trypsin, hyaluronidase and collagenase in rats and mice (Pritzker 1994; van der Kraan et al., 1989; van den Berg et al., 1993), intra-articular injection of specific cytokines, such as interleukin-1 (IL-1), transforming growth factor-β in mice (van de Loo et al., 1994; van den Berg 1995) and oral quinolone (Kato and Ornodera 1988). Osteoarthritic-like changes can also be induced by immobilization of the limb (Troyer 1988) or found spontaneously in mice and guinea pigs (Schunke et al., 1988; Lapveteläinen et al., 1995) The present application preferably relates to changes induced by injection of monosodium iodoacetate.

Injection of monosodium iodoacetate (MIA), a cellular glycolytic inhibitor has been used to induce OA-like changes in the articular cartilage in a variety of animal species with resulting lesioning of the articular cartilage and a marked depletion of proteoglycans as indicated by the loss of safranin O staining (van der Kraan et al., 1992; Gustafson et al., 1992). This model of OA in rodents has been used in the past to examine the pathophysiology of the disease (Kalbhen, 1987; van der Kraan et al., 1992; Guingamp et al., 1997). Because pain is one of the cardinal symptoms of OA, there has been an upsurge in interest recently to examine pain as an end point in this model. All these studies, however, have focused on analyzing hind limb weight bearing distribution following a unilateral induction of OA. This is an index for resting pain behavior as the rats are immobilized during the weight bearing measurement. The present invention uses hind paw grip force assessment in this model as a novel tool to measure use-related pain, a characteristic feature of early stage clinical OA. Patients with OA have pain that typically worsens with activity but improves with rest. If the patient experiences continuous pain at rest, this signifies an increase in severity of the disease. Activity-induced pain is clinically relevant to the early stage OA and measuring pain during activity should be more of a sensitive measure and a better early predictor of pharmacological efficacy than when pain is measured at rest in a preclinical animal model. Recently it has been demonstrated that these resting pain and pain following physical activity observed in OA patients, respond differently to pharmacological interventions. Thus there is a need for preclinical models of OA that incorporate the different aspects of pain behavior and then evaluate the effects of different treatments.

While available models focus on hind paw weight bearing measured as an index for the extent of resting pain behavior in the chemically induced OA model in rats (Kobayashi et al., 2003; Bove et al., 2003, Pomonis et al., 2004)), the present invention uses hind paw grip force assessment in this model as a novel tool to measure use-related pain, a characteristic feature of early stage clinical OA. The preferred animal for this model is a rodent. A rodent may be but is not limited to for example, a rat, a mouse, and a guinea pig.

The detection and measurement of activity-induced pain in the method of the present invention is performed by using simultaneously animals in which ostheoarthritic-like changes have been induced (test animals) and substantially identical naïve animals. To assess the grip force, a commercially available device is used to replicate the clinically observed symptoms of activity-induced pain observed following movement of the load bearing joints like the knee and the hip. Hind limb grip force in the test animals and naïve animals is measured and compared, a significant decrease in the hind limb grip force is considered a measure of the activity-induced pain behavior. The present invention also provides a method for assessing the effects of known analgesics using activity-induced pain in the model of OA described herein. It is intended that test are performed at the same time in animals in which ostheoarthritic-like changes have been induced, and in substantially identical naïve animals. Additionally, the present invention describes a method for assessing and measuring activity-induced pain in the animal model of the present invention, to compare chronic versus acute treatments with drugs used to treat OA. Studies using NSAIDs, tramadol, opiates, and other drugs to treat pain demonstrate that for the same model of pain, different pharmacological effects can be observed depending on the pain end point (activity vs. resting pain) measured and the dosing regimen (acute vs. chronic dosing). These results outline the importance of testing several end points in the same disease model and for a chronic condition, such as chronic pain, the usefulness of testing the effects of repeated dosing.

For evaluating the effects of reference analgesic drugs experiments were performed 20 days after induction of OA. Assessment of hind limb grip force was used as a behavioral measure of activity induced pain in adult OA rodents. Measurements of peak hind limb grip force were conducted by recording the maximum compressive force exerted on the hind limb strain gauge setup, in a commercially available grip force measurement system (Columbus Instruments, Columbus, Ohio). A group of age matched naïve animals were added to each experiment and the data obtained from the different dose groups for the compound being tested were compared to the naïve group.

The method of the present invention can be used also to identify compounds that may be useful in the treatment of OA measuring pain during movement determined by grip force assessment, i.e., a method of screening for a candidate therapeutic agents useful in the early activity-induced pain stage of ostheoarthritis.

The screening can be performed by assessing the effects on the decrement in peak hind limb compressive grip force in test animal versus naïve animals, of an agent following systemic or local administration in osteoarthritic animals, evaluating the degree of return to normalcy in the test animal. The candidate therapeutic agent-induced return to normalcy in the test animal is an indication that the candidate therapeutic agent is a potential therapeutic agent useful in the activity-induced pain stage of osteoarthritis.

It is intended that all the methods using the animal model of the present invention preferentially use a rodent animal, which includes but is not limited to a rat, a mouse, or a guinea pig. It is also intended that the test animal and the naïve animal maybe littermates.

The present invention also includes a method of treating, reversing or limiting the pain of the early stage activity-induced pain in ostheoarthritis with a compound that has been identified using the method described above.

### EXAMPLES

This invention is further described and illustrated by way of the following examples and the experimental detail therein. This section is set forth as an aid to understanding the invention, but is not intended to, nor should it be constructed as, limiting the invention as claimed.

### Example 1

### Time Course of Pain Behavior Assessment

Assessment of hind limb grip force following unilateral injection of MIA was used as a behavioral measure of activity induced pain in adult osteoarthritic rats (body weight 20 days following MIA injection equal to 325-350 g). All experiments have been approved by the Institutional Animal Care and Use Committee (IACUC) at Abbott Laboratories and are in strict accordance with the ethical guidelines laid down by the International Association for the Study of Pain (IASP) (Zimmermann, 1983) for the care and use of laboratory animals. Unilateral knee joint OA was induced in rodents by a single intra-articular (i.a.) injection of sodium monoiodoacetate (MIA) (Sigma-Aldrich, St. Louis, Mo.) (3 mg in 0.05 ml sterile isotonic saline) into the joint cavity under light (1-3%) isoflurane (Hospira, Lake Forest, Ill.) anesthesia using a 26G needle (Pomonis et al., 2004). Following injection, the animals were allowed to recover from the effects of anesthesia (usually 5-10 mins) before returning to their home cages. To maintain uniformity across the study, the right knee joint of each animal was injected with MIA. Animals were euthanized by CO2 inhalation on day 20. The injected knee joints were dissected and immediately fixed in 10% phosphate buffered formalin. The samples were then decalcified in 5% formic acid for 3 days, routinely processed and then embedded in paraffin wax. Frontal histologic sections of the femorotibial joints (6 μm) were prepared, stained with toluidine-blue and examined with a light microscope (Guzman et al., 2003).

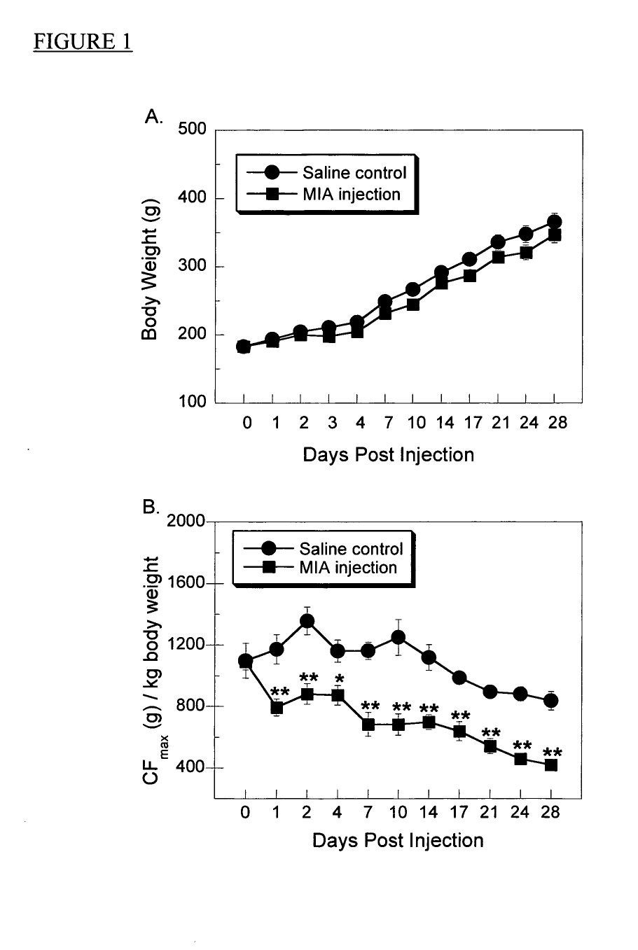

Measurements of peak hind limb grip force were conducted by recording the maximum compressive force exerted on the hind limb strain gauge setup, in a commercially available grip force measurement system (Columbus Instruments, Columbus, Ohio). During testing, each rat was gently restrained by grasping around its rib cage and then allowed to grasp the wire mesh frame (10×12 cm attached to the strain gauge. Animals were then moved in a rostral-to-caudal direction until the grip was broken. Each rat was sequentially tested twice at approximately 2-3 min interval to obtain a raw mean grip force (CFₘₐₓ). This raw mean grip force data was in turn converted to a maximum hindlimb compressive force (CFₘₐₓ) (gram force)/kg body weight for each animal. The assessment of hind limb grip force was conducted for 28 days following the i.a. injection of MIA or saline (control). Baseline measurements were acquired a day prior to the injections of MIA/saline. Additional tests were performed 1, 2, 4, 7, 10, 13, 17, 21, 24, and 28 days following the injections. A group mean±S.E.M. for CFₘₐₓ/kg body weight was calculated at each time point for the 6 animals in the MIA and saline injected groups, respectively. Intra-articular administration of either MIA or saline had no effect on the general health of the animals. The animals in both the saline and MIA injected groups gained body weight normally and there was no statistically significant difference in body weight gain between the two groups (FIG. 1A). The rats with i.a. administration of MIA also exhibited long lasting activity-induced pain behavior throughout the 28-day study period (FIG. 1B). When compared to the saline injected group, the MIA injected group exhibited a significant decrement in hind limb GF (F (21,131)=14.56, P<0.0001). In the MIA injected group, GF decreases from 1090.17±52.85 g before unilateral MIA injection to 420.96±25.07 g at 28 days following injection. When compared to the saline injected group, the MIA injected group exhibited a significant decrement in GF (P<0.05 for treatment, time and time*treatment). In the saline-injected group no difference in GF was observed. Post hoc analysis revealed a significant effect for MIA injection when compared to the saline control from days 1-28 (P<0.05).

### Example 2

### Pharmacological Modulation of the Activity-Induced Pain Behavior (GF)

The analgesic effects of morphine, tramadol, celecoxib, diclofenac, indomethacin, acetaminophen, ibuprofen, gabapentin and lamotrigine were determined on activity induced pain behavior as evaluated by GF assessment, observed 20 days following the i.a. injection of MIA, as described in Example 1.

For evaluating the effects of reference analgesic drugs, the evaluation of hind limb grip force was conducted 20 days following the i.a. injection of MIA. A group of age matched naïve animals were added to each experiment and the data obtained from the different dose groups for the compound being tested were compared to the naïve group. The vehicle control group for each compound being tested was assigned 0% whereas the naïve group was assigned as being 100% (normal). The % effects for each dose group was then expressed as % return to normalcy compared to the naïve group (=[100÷% increase from vehicle for naive group]×% increase from vehicle for treatment group) (% increase from vehicle=[Treatment CFₘₐₓ−Vehicle CFₘₐₓ)/Vehicle CFₘₐₓ]×100). All experiments evaluating drug effects in this model were conducted in a randomized blinded fashion.

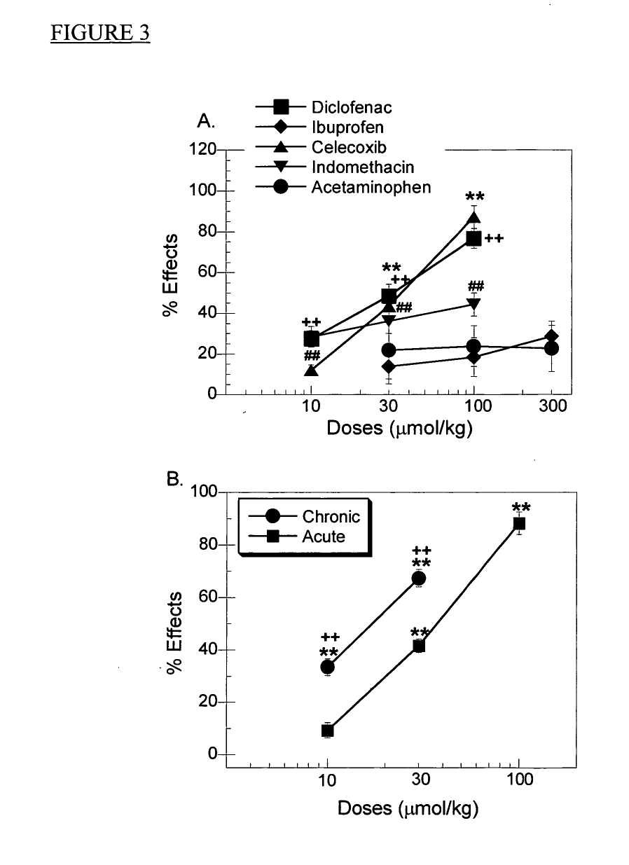

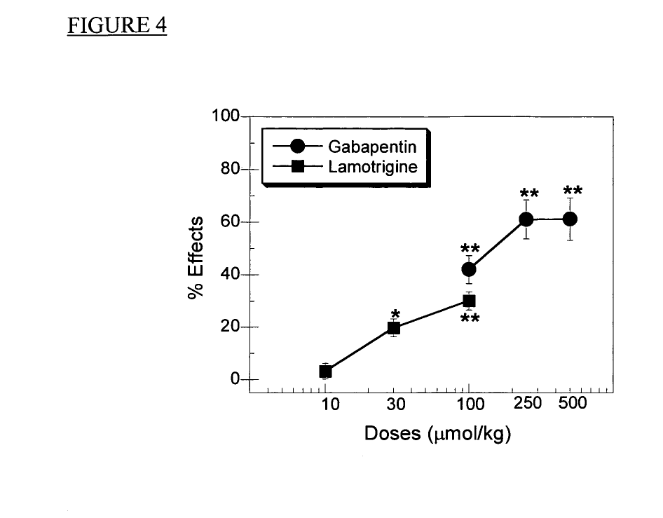

Opioids: Sub-cutaneously administered morphine produces weak effects in this model (34±3% effect at 8 μmol/kg). Vehicle CFₘₐₓ for morphine was 290.16±15.4 g (FIG. 3A). On the other hand, 8 μmol/kg (6 mg/kg) and 13 μmol/kg (10 mg/kg) reduced grip force in naïve rats (non-MIA injected rats) by 13±3% and 52±3% % respectively compared to age-matched naïve untreated rats (CFₘₐₓ=1058.96±30.1 g (FIG. 3B). Intra-peritoneal administered tramadol demonstrates dose-dependent reversal of the decreased grip force (ED₅₀=40 μmol/kg, CI95%=32-54 μmol/kg with 79±5% effect at 100 μmol/kg) (FIG. 3A). Vehicle CFₘₐₓ for tramadol was 248.47±26.9 g (**P<0.01 dose groups vs. vehicle). To further understand the weak effects of morphine in this model, the effects of haloperidol were evaluated when administered in naïve rats. In naïve rats, haloperidol (8±6, 44±9 & 71±2% for the 1, 3 & 10 μmol/kg doses respectively) reduced hind limb GF when compared to the corresponding untreated age-matched group of rats (CFₘₐₓ=1066.17±79.5 g) (FIG. 3B) (**P<0.01 dose groups vs. vehicle).
Acetaminophen and NSAIDs: Among the NSAIDs, p.o. administered celecoxib (ED₅₀=27 μmol/kg, CI95%=16-42 μmol/kg with 87±6% effect at 100 μmol/kg) and p.o. administered diclofenac (ED₅₀=29 μmol/kg, CI95%=22-38 μmol/kg with 77±5% effect at 100 μmol/kg) fully reversed the decreased grip force, whereas weak effects were observed for ibuprofen (29±8% effect at 300 μmol/kg) and indomethacin (44±6% effect at 85 μmol/kg) (FIG. 4A). Vehicle CFₘₐₓ for diclofenac, ibuprofen, indomethacin and celecoxib were 327.16±41.4 g, 400.97±54.7 g, 464.07±32.9 g and 352.02±16.7 g respectively (**P<0.01 dose groups vs. vehicle). Orally administered acetaminophen exhibited no effects (23±11% effect at 300 μmol/kg) (FIG. 4A). Following chronic b.i.d. dosing of the 10 and 30 μmol/kg doses for 12 days, the analgesic efficacy is maintained (33±3.0 and 67±3% effects respectively) which is statistically significant (⁺⁺P<0.01 vs. acute) when compared to the corresponding acutely dosed groups (9±3 and 42±3% effects respectively). A dose of 100 μmol/kg was administered acutely and produced 88±4% serving as a positive control for the study. Vehicle CFₘₐₓ for this study was 336.7±22.1 g (FIG. 4B) (**P<0.01 dose groups vs. vehicle).
Anti-neuropathic pain drugs: Orally (p.o.) administered gabapentin reversed the activity-induced behavior (ED₅₀=159 μmol/kg, CI95%=<100-295.12 μmol/kg with 61±8% effect at 500 μmol/kg) (FIG. 5). Lamotrigine administered orally had weak effects (30±3% effect at 100 μmol/kg) (FIG. 5). Vehicle CFₘₐₓ for gabapentin and lamotrigine were 568.29±13.07 g and 337.75±38.10 g respectively (**P<0.01 dose groups vs. vehicle).

These results demonstrate that for the same model of pain, different pharmacological effects can be observed depending on the pain end point (activity vs. resting pain) measured and the dosing regimen (acute vs. chronic dosing). These results outline the importance of testing several end points in the same disease model and for a chronic condition, such as chronic pain, the usefulness of testing the effects of repeated dosing.

The present invention discloses and claims a novel activity-induced pain behavior in an animal model of OA where it is possible to examine the impact of different pharmacological interventions on this clinically relevant functional measure. The present invention demonstrates the existence of pharmacological differences between activity-induced vs. resting pain behavior, suggesting that the different stages of OA pain viz., activity-induced or pain at rest could require different pain relief approaches.

1) A method of providing a non-human animal model for the detection of activity-induced pain behavior, wherein osteoathritic-like changes have been induced in the non-human animal model, wherein the non-human animal is a rodent.

2) The method of claim 1 wherein the osteoarthritic-like changes have been induced by surgery, immobilization, spontaneously present, chemically-induced and induced by injection of monosodium iodoacetate.

3) The method of claim 2 wherein the osteoarthritic-like changes have been induced by injection of monosodium iodoacetate.

4) The method of claim 1 wherein detection of activity-induced pain behavior is affected by rotorod performance, avoidance behavior, walkway performance and by the peak hindlimb compressive grip force assessment.

5) The method of claim 4 wherein detection of activity-induced pain behavior is affected by the peak hindlimb compressive grip force assessment.

6) The method of claim 5 comprising the steps of:
a) providing, by the method of claim 2, a test animal and a substantially identical naïve animal;
b) measuring peak hind limb compressive grip force (CFₘₐₓ) in the test animal and in the naïve animal;
c) assessing the activity-induced pain behavior by the significant decrement in hind limb compressive grip force in test animal versus naïve animals.

7) (canceled)

8) A method of assessing the effects of analgesic compounds in activity-induced pain behavior using the method of claim 1.

9) A method of assessing and measuring activity-induced pain using the non-human animal model of claim 1, to compare chronic versus acute treatments with drugs used to treat osteoarthritis.

10) A method of screening for a candidate therapeutic agent useful in the early activity-induced pain stage of osteoarthritis using the non-human animal model of claim 1.

11) The method of claim 10 comprising the steps of;
a) providing, by the method of claim 2, a test animal and a substantially identical naïve animal;
b) assessing the activity-induced pain behavior by the significant decrement in peak hind limb compressive grip force in test animal versus naïve animals.
c) administering a candidate agent to the test animal;
d) measuring the effect of the candidate therapeutic agent by measuring the degree of return to normalcy in the test animal;
e) determining that the candidate therapeutic agent-induced return to normalcy in the test animal is an indication that the candidate therapeutic agent is a potential therapeutic agent useful in the activity-induced pain stage of osteoarthritis.

12) The method of claim 11 wherein the test animal and the naïve animal is a rodent.

13) The method of claim 12, wherein the test animal and the naïve animal is a rat.

14) The method of claim 11 wherein the test animal and the naïve animal are littermates.

15) A method of treating, reversing or limiting the pain of the early stage activity-induced pain in ostheoarthritis with a compound that has been identified using the method of claim 11.
